(19) United States
(12) Patent Application Publication
(10) Pub. No.: US 2022/0367058 A1
(43) Pub. Date: Nov. 17, 2022

(54) QUANTUM COMPUTING TECHNIQUES FOR GENERATING EPISTATIC POLYGENIC RISK SCORES

(71) Applicant: Optum, Inc., Minnetonka, MN (US)

(72) Inventors: Matthew R. Versaggi, Plymouth, MN (US); Paul J. Godden, London (GB)

(21) Appl. No.: 17/318,479

(22) Filed: May 12, 2021

(51) Int. Cl. G16H 50/30; G16H 50/20; G06N 10/00

(52) U.S. Cl. CPC G16H 50/30; G16H 50/20; G06N 10/00

(57) ABSTRACT

Various embodiments of the present invention utilize systems, methods, and computer program products that perform health-related predictive data analysis by utilizing an epistatic polygenic risk score generation machine learning model comprises at least one of the following: (i) an epistatic interaction score generation sub-model that is configured to process one or more significant epistatic interaction features for the patient data object that correspond to one or more significant epistatic interactions defined by the epistatic interaction score generation sub-model in order to generate an epistatic interaction score, and (ii) a base polygenic risk score generation sub-model that is configured to process one or more significant genetic variant features for the patient data object that correspond to one or more significant genetic variants defined by the base polygenic risk score generation machine learning model in order to generate a base polygenic risk score.

### BACKGROUND

Various embodiments of the present invention address technical challenges related to performing health-related predictive data analysis. Various embodiments of the present invention address the shortcomings of existing health-related predictive data analysis systems and disclose various techniques for efficiently and reliably performing health-related predictive data analysis.

### BRIEF SUMMARY

In general, embodiments of the present invention provide methods, apparatus, systems, computing devices, computing entities, and/or the like for performing health-related predictive data analysis. Certain embodiments of the present invention utilize systems, methods, and computer program products that perform health-related predictive data analysis by utilizing an epistatic polygenic risk score generation machine learning model comprises at least one of the following: (i) an epistatic interaction score generation sub-model that is configured to process one or more significant epistatic interaction features for the patient data object that correspond to one or more significant epistatic interactions defined by the epistatic interaction score generation sub-model in order to generate an epistatic interaction score, and (ii) a base polygenic risk score generation sub-model that is configured to process one or more significant genetic variant features for the patient data object that correspond to one or more significant genetic variants defined by the base polygenic risk score generation machine learning model in order to generate a base polygenic risk score. Examples of health-related predictive data analysis tasks include genetic predictive data analysis tasks, polygenic predictive data analysis tasks, medical predictive data analysis tasks, behavioral predictive data analysis tasks, and/or medical predictive data analysis tasks.

In accordance with one aspect, a method is provided. In one embodiment, the method comprises: identifying an epistatic polygenic risk score generation machine learning model, wherein: the epistatic polygenic risk score generation machine learning model comprises an epistatic polygenic risk score generation sub-model, the epistatic polygenic risk score generation sub-model is characterized by one or more significant epistatic interactions, and the one or more significant epistatic interactions are determined by: (i) performing a set of quantum superposition operations on a genomic scan data object to generate a group of genome scan qubits, and (ii) performing a set of constructive interference determination operations across the group of genome scan qubits to determine the one or more significant epistatic interactions; generating, using the epistatic polygenic risk score generation machine learning model, the epistatic polygenic risk score, wherein generating the epistatic polygenic risk score using the epistatic polygenic risk score generation machine learning model comprises: (i) processing one or more significant epistatic interaction features corresponding to the one or more significant epistatic interactions using the epistatic polygenic risk score generation sub-model to generate an epistatic interaction score, and (ii) generating the epistatic polygenic risk score based at least in part on the epistatic interaction score, and performing one or more prediction-based actions based at least in part on the epistatic polygenic risk score.

In accordance with another aspect, a computer program product is provided. The computer program product may comprise at least one computer-readable storage medium having computer-readable program code portions stored therein, the computer-readable program code portions comprising executable portions configured to: identify an epistatic polygenic risk score generation machine learning model, wherein: the epistatic polygenic risk score generation machine learning model comprises an epistatic polygenic risk score generation sub-model, the epistatic polygenic risk score generation sub-model is characterized by one or more significant epistatic interactions, and the one or more significant epistatic interactions are determined by: (i) performing a set of quantum superposition operations on a genomic scan data object to generate a group of genome scan qubits, and (ii) performing a set of constructive interference determination operations across the group of genome scan qubits to determine the one or more significant epistatic interactions; generate, using the epistatic polygenic risk score generation machine learning model, the epistatic polygenic risk score, wherein generating the epistatic polygenic risk score using the epistatic polygenic risk score generation machine learning model comprises: (i) processing one or more significant epistatic interaction features corresponding to the one or more significant epistatic interactions using the epistatic polygenic risk score generation sub-model to generate an epistatic interaction score, and (ii) generating the epistatic polygenic risk score based at least in part on the epistatic interaction score, and perform one or more prediction-based actions based at least in part on the epistatic polygenic risk score.

In accordance with yet another aspect, an apparatus comprising at least one processor and at least one memory including computer program code is provided. In one embodiment, the at least one memory and the computer program code may be configured to, with the processor, cause the apparatus to: identify an epistatic polygenic risk score generation machine learning model, wherein: the epistatic polygenic risk score generation machine learning model comprises an epistatic polygenic risk score generation sub-model, the epistatic polygenic risk score generation sub-model is characterized by one or more significant epistatic interactions, and the one or more significant epistatic interactions are determined by: (i) performing a set of quantum superposition operations on a genomic scan data object to generate a group of genome scan qubits, and (ii) performing a set of constructive interference determination operations across the group of genome scan qubits to determine the one or more significant epistatic interactions; generate, using the epistatic polygenic risk score generation machine learning model, the epistatic polygenic risk score, wherein generating the epistatic polygenic risk score using the epistatic polygenic risk score generation machine learning model comprises: (i) processing one or more significant epistatic interaction features corresponding to the one or more significant epistatic interactions using the epistatic polygenic risk score generation sub-model to generate an epistatic interaction score, and (ii) generating the epistatic polygenic risk score based at least in part on the epistatic interaction score, and perform one or more prediction-based actions based at least in part on the epistatic polygenic risk score.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example predictive data analysis computing entity in accordance with some embodiments discussed herein.

FIG. 3 provides an example external computing entity in accordance with some embodiments discussed herein.

FIG. 4 is a flowchart diagram of an example process for generating an epistatic polygenic risk score in accordance with some embodiments discussed herein.

FIG. 5 is a data flow diagram of an example process for generating an epistatic polygenic risk score generation machine learning model in accordance with some embodiments discussed herein.

FIG. 6 is a data flow diagram of an example process for processing a genomic scan data object using an epistatic polygenic risk score generation machine learning model to generate an epistatic polygenic risk score in accordance with some embodiments discussed herein.

FIG. 7 provides an operational example of a predictive output user interface in accordance with some embodiments discussed herein.

### DETAILED DESCRIPTION

Various embodiments of the present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis.

### I. Overview and Technical Advantages

Generating polygenic risk scores that integrate epistatic interactions is a major challenge facing the field of computational genetics. See, e.g., T. McKay & J. Moore, “Why Epistasis Is Important for Tacking Complex Human Disease Genetics,” 6 Genome Medicine 42 (2014) (available online at https://genomemedicine.biomedcentral.com/articles/10.1186/gm561) (“The challenges for detecting epistasis in human populations are threefold . . . Another challenge in the analysis of epistasis is computational, and lies in the number of central processing unit cycles that are required to enumerate all possible combinatorial models. In general, it is not possible to test all possible interactions among more than three SNPs at a time in a genome-wide scan. A final challenge is interpretation. High-order interactions with non-additive effects can be difficult to comprehend statistically and perhaps even harder to tie back to biology. Designing combinatorial experiments to validate epistasis models might be more difficult than the analytical challenges.”).

Various embodiments of the present invention address the above-noted challenges associated with computational complexity of integrating epistatic interactions into polygenic risk score computation by utilizing quantum computing techniques. For example, in some embodiments, an epistatic polygenic risk score generation machine learning model is characterized by a set of significant epistatic interactions, where the set of significant epistatic interactions are determined by: (i) performing a set of quantum superposition operations on a genomic scan data object to generate a group of genome scan qubits, and (ii) performing a set of constructive interference determination operations across the group of genome scan qubits to determine the one or more significant epistatic interactions. By utilizing the noted techniques, various embodiments of the present invention enable reducing the amount of time and/or computational operations needed to detect significant epistatic interactions and integrate those significant epistatic integrations into polygenic risk score generations. In some embodiments, once identified and integrated into polygenic risk score generation models, epistatic interactions can be computed in an efficient manner, such as in linear time.

Moreover, various embodiments of the present invention address technical challenges related to improving computational efficiency and/or operational reliability of performing health-related predictive data analysis. Health-related predictive data analysis systems face substantial challenges because they are tasked with integrating predictive insights related to physiological diversity across the human population (e.g., the genetic diversity of human genome across humans). Because of the noted challenges, various existing predictive data analysis solutions are either highly ineffective and/or too computationally costly. To address the noted concerns related to computational efficiency and/or operational reliability of performing health-related predictive data analysis, various embodiments of the present invention introduce innovative techniques for generating computationally efficient epistatic polygenic risk scores that enable performing health condition modeling in a reliable but efficient manner.

### II. Definitions

The term “epistatic polygenic risk score” may refer to a data object that is configured to describe a value that in turn describes an inferred risk that a patient data object describing a patient may be predicted to in the future suffer from a disease (i.e., condition) described by a disease data object. The epistatic polygenic risk score may be determined based at least in part on an epistatic interaction score that describes how much n-groupings of location-specific nucleobase features described in defined loci of a genomic scan data object of the patient data object suggest that the patient associated with the patient data object may be predicted to in the future suffer from the disease described by the disease data object. In some embodiments, the epistatic polygenic risk score is determined based at least in part on both of: (i) a base polygenic risk score that describes how much individual genetic variant features described by the genomic scan data object of the patient data object suggest that the patient associated with the patient data object may be predicted to in the future suffer from the disease described by the disease data object, and (ii) an epistatic interaction score that describes how much n-groupings of location-specific nucleobase features described in defined loci of a genomic scan data object of the patient data object suggest that the patient associated with the patient data object may be predicted to in the future suffer from the disease described by the disease data object. For example, in some embodiments, the epistatic polygenic risk score is determined based at least in part on addition of the base polygenic risk score and the epistatic interaction score. As another example, in some embodiments, the epistatic polygenic risk score is determined based at least in part on a defined linear combination of the base polygenic risk score and the epistatic interaction score.

The term “base polygenic risk score” may refer to a data object that is configured to describe a value that in turn describes an inferred risk that a patient data object describing a patient may be predicted to in the future suffer from a disease described by a disease data object, where the inferred risk is determined based at least in part on individual genetic variant features described by the genomic scan data object of the patient data object. In some embodiments, the base polygenic risk score describes how much individual genetic variant features described by the genomic scan data object of the patient data object suggest that the patient associated with the patient data object may be predicted to in the future suffer from the disease described by the disease data object. In some embodiments, the base polygenic risk score is a conventional Polygenic Risk Score value that is determined using a Polygenic Risk Score equation for the disease data object, where the Polygenic Risk Score equation may be determined by performing methods such as linkage disequilibrium (LD) adjustment, Beta shrinkage, and P-value threshold across data describing correlations between genetic variants and disease occurrences as determined based at least in part on results of a genome-wide association study data. In some embodiments, the base polygenic risk score describes whether the genomic scan data object associated with the patient data object describes each of a set of significant genetic variants as described by a base polygenic risk score generation sub-model of an epistatic polygenic risk score generation machine learning model.

The term “epistatic interaction score” may refer to a data object that is configured to describe a value that in turn describes an inferred risk that a patient data object describing a patient may be predicted to in the future suffer from a disease described by a disease data object, where the inferred risk is determined based at least in part on epistatic interactions across re-groupings of location-specific nucleobase features described in defined loci of a genomic scan data object of the patient data object. In some embodiments, the epistatic interaction score describes how much n-groupings of location-specific nucleobase features described in defined loci of a genomic scan data object of the patient data object suggest that the patient associated with the patient data object may be predicted to in the future suffer from the disease described by the disease data object. In some embodiments, the epistatic interaction score is determined based at least in part on whether the genomic scan data object associated with the patient data object describes each of a set of significant epistatic interactions, where the set of significant epistatic interactions may be defined by an epistatic interaction score generation sub-model of an epistatic polygenic risk score generation machine learning model.

The term “epistatic polygenic risk score generation machine learning model” may refer to a data object that is configured to describe parameters, hyper-parameters, and/or defined operations of a model (e.g., a linear model or a non-linear model) that is configured to generate an epistatic polygenic risk score for a patient data object in relation to a corresponding disease data object based at least in part on whether a genomic scan data object for the patient data object describes presence of a set of significant epistatic interactions. For example, in some embodiments, the epistatic polygenic risk score generation machine learning model is configured to generate an epistatic polygenic risk score for a patient data object in relation to a disease data object describing the cystic fibrosis disease based at least in part on whether a genomic scan data object for the patient data object describes presence of a set of significant epistatic interactions. In some embodiments, the epistatic polygenic risk score generation machine learning model comprises at least one of the following: (i) an epistatic interaction score generation sub-model that is configured to process one or more significant epistatic interaction features for the patient data object that correspond to one or more significant epistatic interactions defined by the epistatic interaction score generation sub-model in order to generate an epistatic interaction score, and (ii) a base polygenic risk score generation sub-model that is configured to process one or more significant genetic variant features for the patient data object that correspond to one or more significant genetic variants defined by the base polygenic risk score generation machine learning model in order to generate a base polygenic risk score. In some embodiments, the epistatic polygenic risk score generation machine learning model comprises a equation having the form rₜₒₜₐₗ=θ₁r_{conv}Δθ₂rₑₚᵢₛₜₐₛᵢₛ, where rₜₒₜₐₗ is the epistatic polygenic risk score, r_{conv} is the base polygenic risk score, rₑₚᵢₛₜₐₛᵢₛ is the base epistatic interaction score, θ₁ and θ₂ are the respective weights for r_{conv} and rₑₚᵢₛₜₐₛᵢₛ (e.g., as determined using a maximum likelihood estimator for a normally distributed nonlinear implicit relationship defined by the epistatic polygenic risk score generation machine learning model, for example using the maximum likelihood estimators described in G. R. Dolby and S. Lipton, Maximum Likelihood Estimation of the General Nonlinear Functional Relationship with Replicated Observations and Correlated Errors, Biometrika, Vol. 59, No. 1 (April, 1972), pp. 121-129 (available online at https://projecteuclid.org/download/pdf_1/euclid.aos/1176350696)), and the operator A may have an algebraic form (e.g., may be one of addition, subtraction, multiplication , or division), which will be determined by iteratively cycling through the four possibilities and checking against hold-out data (e.g., positive cases of the disease in question obtained from the genome-wide association study result data).

The term “epistatic interaction score generation sub-model” may refer to a component of an epistatic polygenic risk score generation machine learning model that is configured to process one or more significant epistatic interaction features corresponding to one or more significant epistatic interactions to generate an epistatic interaction score for a patient data object. The epistatic polygenic risk score generation machine learning model may be characterized by a set of significant epistatic interactions, where the set of significant epistatic interactions may be determined by: (i) performing a set of quantum superposition operations on a genomic scan data object to generate a group of genome scan qubits, and (ii) performing a set of constructive interference determination operations across the group of genome scan qubits to determine the one or more significant epistatic interactions. In some embodiments, the epistatic interaction score generation sub-model defines an interaction weight for each significant epistatic interaction, where the interaction weight for a significant epistatic interaction may be determined by generating a maximum likelihood estimation for the significant epistatic interaction using a maximum likelihood estimator for a normally distributed nonlinear implicit relationship defined by the epistatic polygenic risk score generation machine learning model, for example using the maximum likelihood estimators described in G. R. Dolby and S. Lipton, Maximum Likelihood Estimation of the General Nonlinear Functional Relationship with Replicated Observations and Correlated Errors, Biometrika, Vol. 59, No. 1 (April, 1972), pp. 121-129 (available online at https://projecteuclid.org/download/pdf_1/euclid.aos/1176350696).

The term “polygenic risk score generation sub-model” may refer to a component of an epistatic polygenic risk score generation machine learning model that is configured to process one or more significant genetic variant features corresponding to one or more significant genetic variants using the base polygenic risk score generation sub-model to generate a base polygenic risk score. In some embodiments, the polygenic risk score generation sub-model is characterized by a set of significant genetic variants that are determined using a statistical analysis of genome-wide association study result data using at least one of LD adjustment, Beta shrinkage, and P-value threshold across data describing correlations between genetic variants and disease occurrences as determined based at least in part on results of a genome-wide association study data. In some embodiments, the polygenic risk generation sub-model comprises a Polygenic Risk Score equation for the disease data object, where the Polygenic Risk Score equation may be determined by performing LD adjustment, Beta shrinkage, and P-value threshold across data describing correlations between genetic variants and disease occurrences as determined based at least in part on results of a genome-wide association study data. In some embodiments, the polygenic risk generation sub-model comprises an equation that is configured to generate a conventional Polygenic Risk Score.

The term “quantum superposition operation” may refer to a data object that is configured to describe a computer-implemented operation that is configured to map a defined unit of genomic data in a genomic scan data object to a qubit representation of the defined unit (referred to herein as genome scan qubit). In some embodiments, a quantum superposition operation is configured to transform the nucleobase feature represented by a locus of a genomic scan data object to a qubit representation of the nucleobase feature. In some of the noted embodiments, a quantum superposition operation is configured to transform the nucleobase feature represented by a locus of a genomic scan data object to a qubit representation of the nucleobase feature by applying a Hadamard logic gate transformation characterized by a Hadamard gate to the nucleobase feature. In some embodiments, a quantum superposition operation is configured to transform the nucleobase features represented by a base pair of a genomic scan data object to a qubit representation of the pairwise nucleobase features. In some of the noted embodiments, a quantum superposition operation is configured to transform the nucleobase features represented by a base pair a genomic scan data object to a qubit representation of the pairwise nucleobase features by applying a Hadamard logic gate transformation characterized by a Hadamard gate to the pairwise nucleobase features. In some embodiments, performing the set of quantum superposition operations comprises: identifying a group of loci associated with the genomic scan data object; for each locus, determining, based at least in part on applying a Hadamard quantum logic gate transformation of a nucleobase feature described by the locus, a superposed representation; and determining the group of genome scan qubits based at least in part on each superposed representation. In some embodiments, performing the set of quantum superposition operations comprises: identifying a group of base pairs associated with the genomic scan data object; for each base pair, determining, based at least in part on applying a Hadamard quantum logic gate transformation of a pair of nucleobase features described by the base pair, a superposed representation; and determining the group of genome scan qubits based at least in part on each superposed representation.

The term “constructive interference determination operation” may refer to a data object that is configured to describe a computer-implemented operation that is configured to determine, for each defined epistatic interaction between a defined group of two or more location-specific nucleobase features, whether the defined epistatic interaction corresponds to a constructive quantum interference or a destructive quantum interference. In some embodiments, as part of a constructive interference determination operation that is associated with a defined epistatic interaction, if the defined epistatic interaction is deemed a beneficial epistatic occurrence, it is modeled by a constructive quantum interference, while if the defined epistatic interaction is deemed a deleterious epistatic interaction, it is modeled by a destructive quantum interference. For example, in some embodiments, when performing a constructive interference determination operation with respect to an epistatic interaction that is characterized by presence of a guanine (G) nucleobase in a first defined locus and an adenine (A) nucleobase in a second defined locus, if the presence of the G nucleobase in the first defined locus along with the simultaneous presence of the A nucleobase in the second defined locus is deemed to increase the risk of a disease occurrence, then the epistatic interaction may be modeled using a constructive quantum interference; otherwise, the epistatic interaction may be modeled using a destructive quantum interference. In some embodiments, a constructive interference determination operation is configured to process the qubits corresponding to a corresponding epistatic interaction in the following manner to determine whether the epistatic interaction corresponds to a constructive quantum interference or a destructive quantum interference: perform quantum computing operations are performed to determine the output of an interference between the qubits corresponds to the corresponding epistatic interaction and then determine whether the output is a constructive quantum interference or a destructive quantum interference. For example, in some embodiments, when performing a constructive interference determination operation with respect to an epistatic interaction that is characterized by presence of a thymine (T) nucleobase in a first defined locus and a guanine (G) nucleobase in a second defined locus, quantum computing operations are performed to determine the output of an interference between a qubit representation of the T nucleobase in the first defined locus and a qubit representation of the G nucleobase in the second defined locus, and then the noted output is used to determine whether the output is a constructive quantum interference or a destructive quantum interference. In some embodiments, quantum computing operations configured to perform interference between qubits use amplitudes (e.g., special combination of phases and magnitudes), which enable encoding additional information into qubits and further enable creating quantum digital and quantum phase logic that leverages interference for solving large complex problems.

The term “maximal epistatic interaction order hyper-parameter” may refer to data object that is configured to describe the sizes of location-specific nucleobase features involved in epistatic interactions that may be deemed to be significant epistatic interactions for an epistatic interaction score generation sub-model of an epistatic polygenic risk score generation machine learning model. For example, if the maximal epistatic interaction order hyper-parameter is three, then the significant epistatic interactions may include pairwise epistatic interactions between pairs of location-specific nucleobase features (e.g., a pair of location-specific nucleobase features that is characterized by presence of a guanine (G) nucleobase in a first defined locus and an adenine (A) nucleobase in a second defined locus), as well as triplet epistatic interactions between triplets of location-specific nucleobase features (e.g., a triplet of location-specific nucleobase features that is characterized by presence of a thymine (T) nucleobase in a first defined locus, a guanine (G) nucleobase in a second defined locus, and a cytosine (C) nucleobase in a third defined locus). In some embodiments, when determining the significant epistatic interactions for an epistatic interaction score generation sub-model of an epistatic polygenic risk score generation machine learning model, a training engine is configured to map defined groupings of n location-specific nucleobase features to a quantum space in order to perform constructive interference determination operations on each of the groupings, where n={1, . . . , h} and where h is the maximal epistatic interaction order hyper-parameter.

### III. Computer Program Products, Methods, and Computing Entities

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations. Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. Exemplary System Architecture

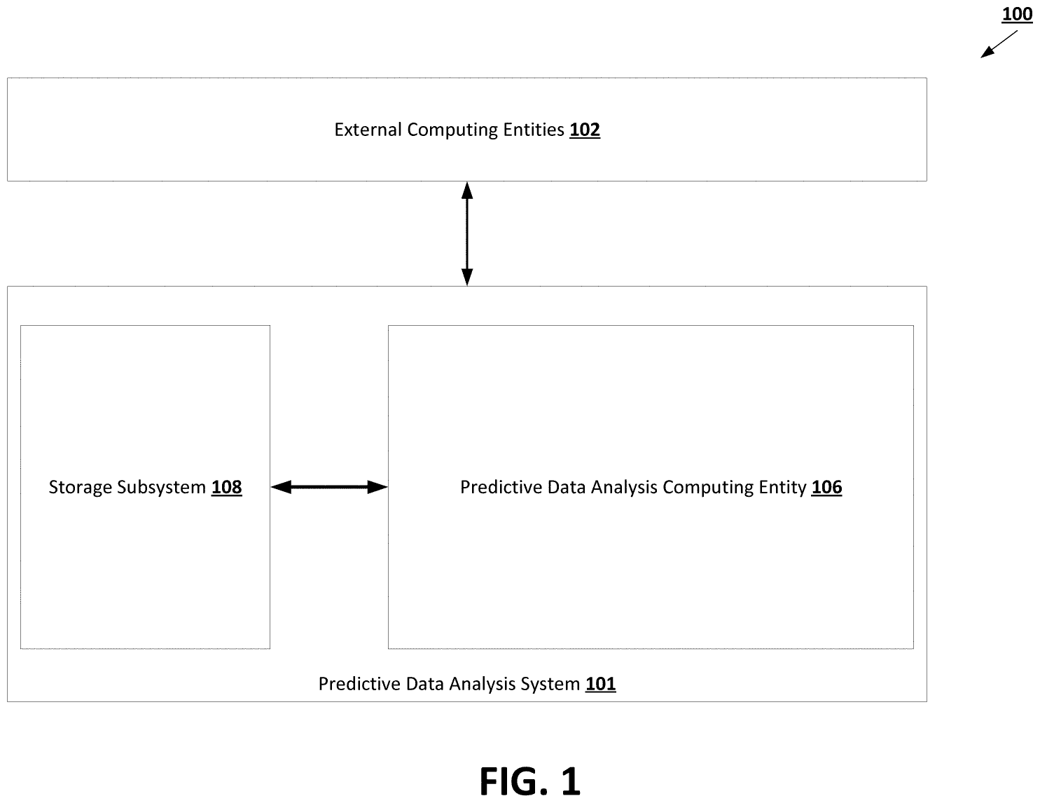

FIG. 1 is a schematic diagram of an example architecture 100 for performing health-related predictive data analysis. The architecture 100 includes a predictive data analysis system 101 configured to receive health-related predictive data analysis requests from external computing entities 102, process the predictive data analysis requests to generate health-related risk predictions, provide the generated health-related risk predictions to the external computing entities 102, and automatically perform prediction-based actions based at least in part on the generated polygenic risk score predictions. Examples of health-related predictions include genetic risk predictions, polygenic risk predictions, medical risk predictions, clinical risk predictions, behavioral risk predictions, and/or the like.

In some embodiments, predictive data analysis system 101 may communicate with at least one of the external computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The predictive data analysis system 101 may include a predictive data analysis computing entity 106 and a storage subsystem 108. The predictive data analysis computing entity 106 may be configured to receive health-related predictive data analysis requests from one or more external computing entities 102, process the predictive data analysis requests to generate the polygenic risk score predictions corresponding to the predictive data analysis requests, provide the generated polygenic risk score predictions to the external computing entities 102, and automatically perform prediction-based actions based at least in part on the generated polygenic risk score predictions.

The storage subsystem 108 may be configured to store input data used by the predictive data analysis computing entity 106 to perform health-related predictive data analysis as well as model definition data used by the predictive data analysis computing entity 106 to perform various health-related predictive data analysis tasks. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Predictive Data Analysis Computing Entity

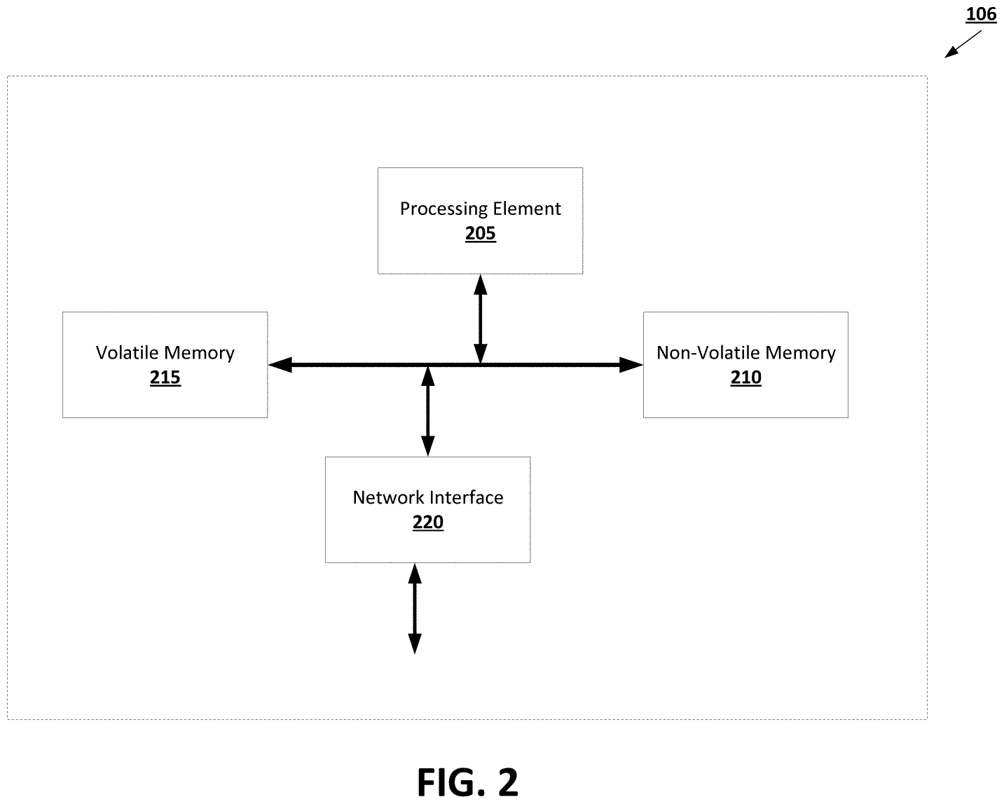

FIG. 2 provides a schematic of a predictive data analysis computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the predictive data analysis computing entity 106 may include or be in communication with one or more processing elements 205 (also referred to as processors, processing circuitry, and/or similar terms used herein interchangeably) that communicate with other elements within the predictive data analysis computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the predictive data analysis computing entity 106 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or memory media 210, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the predictive data analysis computing entity 106 may further include or be in communication with volatile media (also referred to as volatile storage, memory, memory storage, memory circuitry and/or similar terms used herein interchangeably). In one embodiment, the volatile storage or memory may also include one or more volatile storage or memory media 215, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 205 and operating system.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1X (1xRTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may include or be in communication with one or more input elements, such as a keyboard input, a mouse input, a touch screen/display input, motion input, movement input, audio input, pointing device input, joystick input, keypad input, and/or the like. The predictive data analysis computing entity 106 may also include or be in communication with one or more output elements (not shown), such as audio output, video output, screen/display output, motion output, movement output, and/or the like.

### Exemplary External Computing Entity

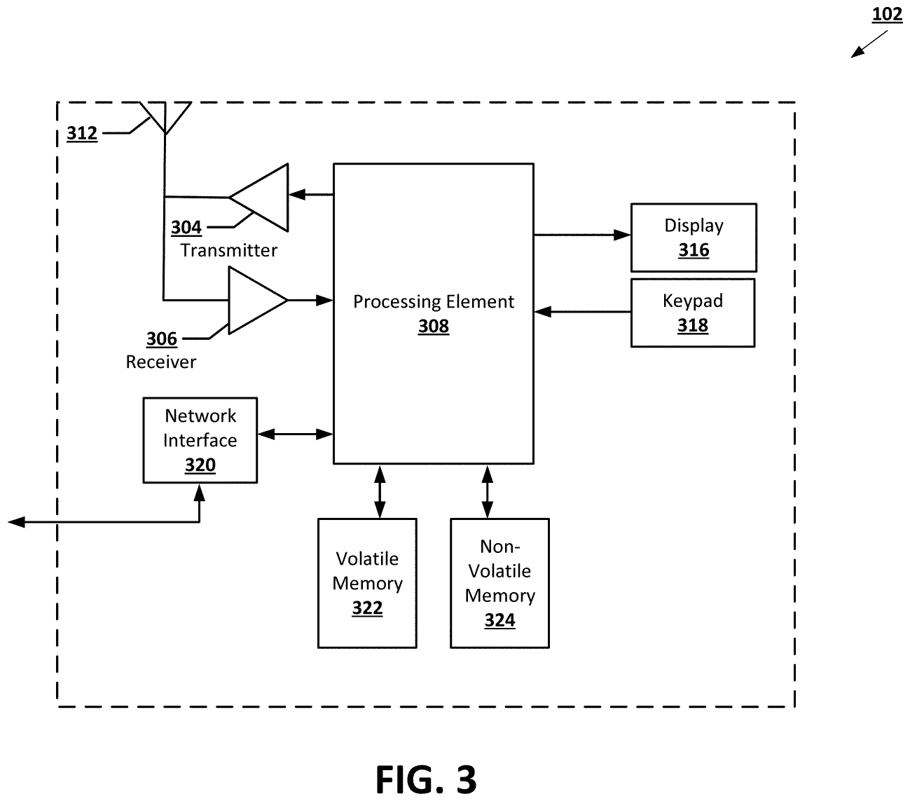

FIG. 3 provides an illustrative schematic representative of an external computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. External computing entities 102 can be operated by various parties. As shown in FIG. 3, the external computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the external computing entity 102 may be capable of operating with one or more air interface standards, communication protocols, modulation types, and access types. More particularly, the external computing entity 102 may operate in accordance with any of a number of wireless communication standards and protocols, such as those described above with regard to the predictive data analysis computing entity 106. In a particular embodiment, the external computing entity 102 may operate in accordance with multiple wireless communication standards and protocols, such as UMTS, CDMA2000, 1xRTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the external computing entity 102 may operate in accordance with multiple wired communication standards and protocols, such as those described above with regard to the predictive data analysis computing entity 106 via a network interface 320.

Via these communication standards and protocols, the external computing entity 102 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 102 can also download changes, add-ons, and updates, for instance, to its firmware, software (e.g., including executable instructions, applications, program modules), and operating system.

According to one embodiment, the external computing entity 102 may include location determining aspects, devices, modules, functionalities, and/or similar words used herein interchangeably. For example, the external computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the external computing entity's 102 position in connection with a variety of other systems, including cellular towers, Wi-Fi access points, and/or the like. Similarly, the external computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The external computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the external computing entity 102 to interact with and/or cause display of information/data from the predictive data analysis computing entity 106, as described herein. The user input interface can comprise any of a number of devices or interfaces allowing the external computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the external computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The external computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the external computing entity 102. As indicated, this may include a user application that is resident on the entity or accessible through a browser or other user interface for communicating with the predictive data analysis computing entity 106 and/or various other computing entities.

In another embodiment, the external computing entity 102 may include one or more components or functionality that are the same or similar to those of the predictive data analysis computing entity 106, as described in greater detail above. As will be recognized, these architectures and descriptions are provided for exemplary purposes only and are not limiting to the various embodiments.

In various embodiments, the external computing entity 102 may be embodied as an artificial intelligence (AI) computing entity, such as an Amazon Echo, Amazon Echo Dot, Amazon Show, Google Home, and/or the like. Accordingly, the external computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. Exemplary System Operations

As described above, generating polygenic risk scores that integrate epistatic interactions is a major challenge facing the field of computational genetics. See, e.g., T. McKay & J. Moore, “Why Epistasis Is Important for Tacking Complex Human Disease Genetics,” 6 Genome Medicine 42 (2014) (available online at https://genomemedicine.biomedcentral.com/articles/10.1186/gm561) (“The challenges for detecting epistasis in human populations are threefold . . . Another challenge in the analysis of epistasis is computational, and lies in the number of central processing unit cycles that are required to enumerate all possible combinatorial models. In general, it is not possible to test all possible interactions among more than three SNPs at a time in a genome-wide scan. A final challenge is interpretation. High-order interactions with non-additive effects can be difficult to comprehend statistically and perhaps even harder to tie back to biology. Designing combinatorial experiments to validate epistasis models might be more difficult than the analytical challenges.”). Various embodiments of the present invention address the above-noted challenges associated with computational complexity of integrating epistatic interactions into polygenic risk score computation by utilizing quantum computing techniques. For example, in some embodiments, an epistatic polygenic risk score generation machine learning model is characterized by a set of significant epistatic interactions, where the set of significant epistatic interactions are determined by: (i) performing a set of quantum superposition operations on a genomic scan data object to generate a group of genome scan qubits, and (ii) performing a set of constructive interference determination operations across the group of genome scan qubits to determine the one or more significant epistatic interactions. By utilizing the noted techniques, various embodiments of the present invention enable reducing the amount of time and/or computational operations needed to detect significant epistatic interactions and integrate those significant epistatic integrations into polygenic risk score generations. In some embodiments, once identified and integrated into polygenic risk score generation models, epistatic interactions can be computed in an efficient manner, such as in linear time.

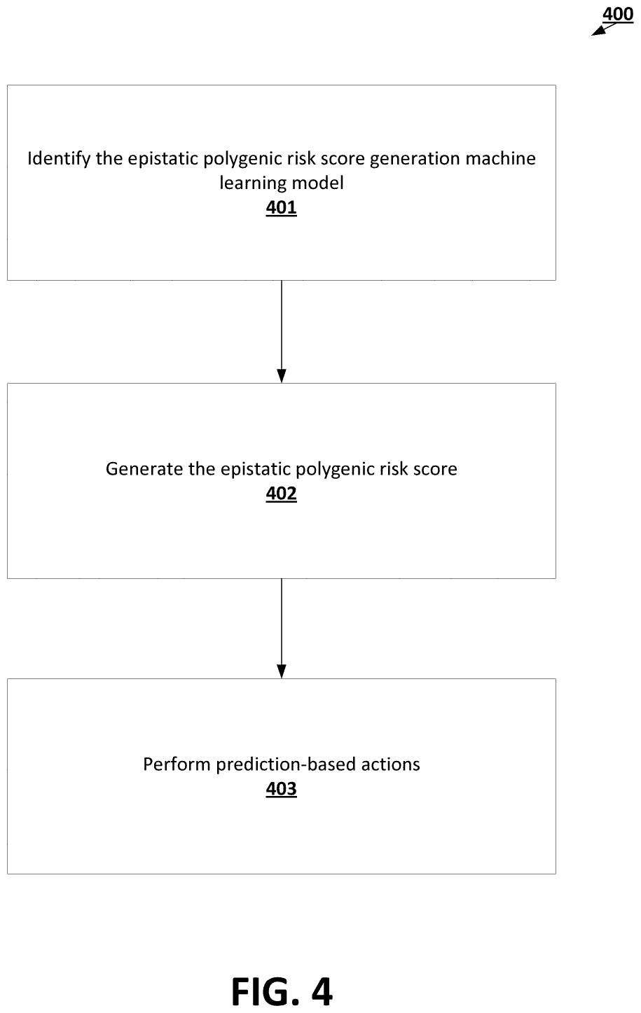

FIG. 4 is a flowchart diagram of an example process 400 for generating an epistatic polygenic risk score. Via the various steps/operations of the process 400, the predictive data analysis computing entity 106 can efficiently generate risk scores that integrate both contribution of singular genetic variants to disease probability and contribution of groupings of genetic variants in defined loci to disease probability.

The process 400 begins at step/operation 401 when the predictive data analysis computing entity 106 identifies the epistatic polygenic risk score generation machine learning model. In some embodiments, the predictive data analysis computing entity 106 retrieves the epistatic polygenic risk score generation machine learning model from the storage subsystem 108. In some embodiments, the predictive data analysis computing entity 106 receives configuration data associated with the epistatic polygenic risk score generation machine learning model from an external computing entity 102. In some embodiments, the predictive data analysis computing entity 106 performs one or more training operations in order to generate the epistatic polygenic risk score generation machine learning model.

In some embodiments, the epistatic polygenic risk score generation machine learning model is configured to generate an epistatic polygenic risk score for a patient data object in relation to a corresponding disease data object based at least in part on whether a genomic scan data object for the patient data object describes presence of a set of significant epistatic interactions. For example, in some embodiments, the epistatic polygenic risk score generation machine learning model is configured to generate an epistatic polygenic risk score for a patient data object in relation to a disease data object describing the cystic fibrosis disease based at least in part on whether a genomic scan data object for the patient data object describes presence of a set of significant epistatic interactions. In some embodiments, the epistatic polygenic risk score generation machine learning model comprises at least one of the following: (i) an epistatic interaction score generation sub-model that is configured to process one or more significant epistatic interaction features for the patient data object that correspond to one or more significant epistatic interactions defined by the epistatic interaction score generation sub-model in order to generate an epistatic interaction score, and (ii) a base polygenic risk score generation sub-model that is configured to process one or more significant genetic variant features for the patient data object that correspond to one or more significant genetic variants defined by the base polygenic risk score generation machine learning model in order to generate a base polygenic risk score.

In some embodiments, the epistatic polygenic risk score generation machine learning model comprises a equation having the form rₜₒₜₐₗ=θ₁r_{conv}Δθ₂rₑₚᵢₛₜₐₛᵢₛ, where rₜₒₜₐₗ is the epistatic polygenic risk score, r_{conv} is the base polygenic risk score, rₑₚᵢₛₜₐₛᵢₛ is the base epistatic interaction score, θ₁ and θ₂ are the respective weights for r_{conv} and rₑₚᵢₛₜₐₛᵢₛ (e.g., as determined using a maximum likelihood estimator for a normally distributed nonlinear implicit relationship defined by the epistatic polygenic risk score generation machine learning model, for example using the maximum likelihood estimators described in G. R. Dolby and S. Lipton, Maximum Likelihood Estimation of the General Nonlinear Functional Relationship with Replicated Observations and Correlated Errors, Biometrika, Vol. 59, No. 1 (April, 1972), pp. 121-129 (available online at https://projecteuclid.org/download/pdf_1/euclid.aos/1176350696)), and the operator A may have an algebraic form (e.g., may be one of addition, subtraction, multiplication , or division), which will be determined by iteratively cycling through the four possibilities and checking against hold-out data (e.g., positive cases of the disease in question obtained from the genome-wide association study result data).

In some embodiments, the epistatic polygenic risk score generation machine learning model comprises an epistatic polygenic risk generation sub-model that is configured to process one or more significant epistatic interaction features corresponding to one or more significant epistatic interactions to generate an epistatic interaction score for a patient data object. The epistatic polygenic risk score generation machine learning model may be characterized by a set of significant epistatic interactions, where the set of significant epistatic interactions may be determined by: (i) performing a set of quantum superposition operations on a genomic scan data object to generate a group of genome scan qubits, and (ii) performing a set of constructive interference determination operations across the group of genome scan qubits to determine the one or more significant epistatic interactions. In some embodiments, the epistatic interaction score generation sub-model defines an interaction weight for each significant epistatic interaction, where the interaction weight for a significant epistatic interaction may be determined by generating a maximum likelihood estimation for the significant epistatic interaction using a maximum likelihood estimator for a normally distributed nonlinear implicit relationship defined by the epistatic polygenic risk score generation machine learning model, for example using the maximum likelihood estimators described in G. R. Dolby and S. Lipton, Maximum Likelihood Estimation of the General Nonlinear Functional Relationship with Replicated Observations and Correlated Errors, Biometrika, Vol. 59, No. 1 (April, 1972), pp. 121-129 (available online at https://projecteuclid.org/download/pdf_1/euclid.aos/1176350696).

In some embodiments, the epistatic polygenic risk score generation machine learning model comprises a base polygenic risk generation sub-model that is configured to process one or more significant genetic variant features corresponding to one or more significant genetic variants using the base polygenic risk score generation sub-model to generate a base polygenic risk score. In some embodiments, the polygenic risk score generation sub-model is characterized by a set of significant genetic variants that are determined using a statistical analysis of genome-wide association study result data using at least one of LD adjustment, Beta shrinkage, and P-value threshold across data describing correlations between genetic variants and disease occurrences as determined based at least in part on results of a genome-wide association study data. In some embodiments, the polygenic risk generation sub-model comprises a Polygenic Risk Score equation for the disease data object, where the Polygenic Risk Score equation may be determined by performing linkage disequilibrium (LD) adjustment, Beta shrinkage, and P-value threshold across data describing correlations between genetic variants and disease occurrences as determined based at least in part on results of a genome-wide association study data. In some embodiments, the polygenic risk generation sub-model comprises an equation that is configured to generate a conventional Polygenic Risk Score.

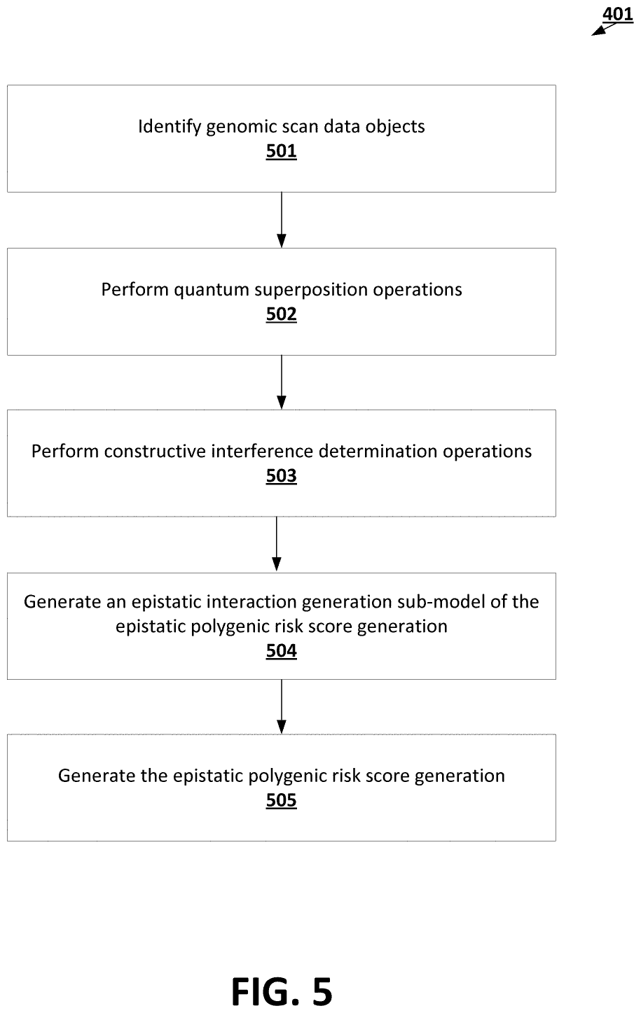

In some embodiments, step/operation 401 may be performed in accordance with the process that is depicted in FIG. 5, which is an example process for generating an epistatic polygenic risk score generation machine learning model using genomic scan data objects associated with a set of patients. Although the process that is depicted in FIG. 5 herein is described as being performed by the predictive data analysis computing entity 106, a person of ordinary skill in the relevant technology will recognize that the noted process may be performed by a training computing entity that is different from and/or remote from the predictive data analysis computing entity 106 and/or from the predictive data analysis system 101.

The process that is depicted in FIG. 5 begins at step/operation 501 when the predictive data analysis computing entity 106 identifies genomic scan data objects, where each genomic scan data object is associated with a value that describes whether a corresponding patient data object is deemed to suffer from a disease data object describing a medical condition of interest. For example, the genomic scan data objects may describe genetic composition of a set of individuals, where each data object is assigned a one value if the corresponding individual is deemed to suffer from a condition of interest and a zero value otherwise. In some embodiments, the genomic scan data objects are retrieved from the results of one or more genome-wide association studies. In some embodiments, if formal genome wide association study results are not available for a condition of interest, then public-domain summary statistics, such as from https://www.ebi.ac.uk/gwas/efotraits/EFO_0001359, are acquired for analysis. In some embodiments, if study data for the condition of interest are not available, the predictive data analysis computing entity 106 performs operations configured to source the raw data, e.g., from LD Hub (http://ldsc.broadinstitute.org/). In some embodiments, each genomic scan data object is a FASTQ file.

In some embodiments, it is assumed that the polygenic risk data base data are in the form of case-control cohorts for the selected disease to be risk-scored. Furthermore, in some embodiments, it is assumed that that the sizes of the cohorts are minimally-sufficient for the statistical power needed for signal detection (e.g., both case and control cohorts have at least 5,000 participants). In some embodiments, it is assumed that the predictive data analysis computing entity 106 has access to the raw FASTQ data, as well as the summary statistics, as the latter enables generation of a base polygenic risk score for the disease of interest via a conventional method, as the weights will be available from the genome-wide association study result data. Aspects of the present invention utilize the FASTQ data for the epistasis calculations, where the epistasis calculations may be performed on a quantum computing platform.

At step/operation 502, the predictive data analysis computing entity 106 performs a set of quantum superposition operations on each genomic scan data object to generate a group of genome scan qubits for the genomic scan data object. In some embodiments, for each single loci in each of the assumed 10,000 genomes in an overall cohort, the predictive data analysis computing entity 106 implements a Hadamard gate to leverage quantum superposition. At each individual locus (e.g., starting at chromosome #1, position #1), the predictive data analysis computing entity 106 may set a Hadamard gate for that G, C, A or T, and then repeat for each other locus for all FASTQ files in the cohort. In quantum information processing, the Hadamard transformation, more often called Hadamard Gate in this context, takes a qubit and puts it into superposition where a that qubit can represent multiple values simultaneously during quantum computation.

In some embodiments, each quantum superposition operation is configured to map a defined unit of genomic data in a genomic scan data object to a qubit representation of the defined unit (referred to herein as genome scan qubit). In some embodiments, a quantum superposition operation is configured to transform the nucleobase feature represented by a locus of a genomic scan data object to a qubit representation of the nucleobase feature. In some of the noted embodiments, a quantum superposition operation is configured to transform the nucleobase feature represented by a locus of a genomic scan data object to a qubit representation of the nucleobase feature by applying a Hadamard logic gate transformation characterized by a Hadamard gate to the nucleobase feature. In some embodiments, a quantum superposition operation is configured to transform the nucleobase features represented by a base pair of a genomic scan data object to a qubit representation of the pairwise nucleobase features. In some of the noted embodiments, a quantum superposition operation is configured to transform the nucleobase features represented by a base pair a genomic scan data object to a qubit representation of the pairwise nucleobase features by applying a Hadamard logic gate transformation characterized by a Hadamard gate to the pairwise nucleobase features. In some embodiments, performing the set of quantum superposition operations comprises: identifying a group of loci associated with the genomic scan data object; for each locus, determining, based at least in part on applying a Hadamard quantum logic gate transformation of a nucleobase feature described by the locus, a superposed representation; and determining the group of genome scan qubits based at least in part on each superposed representation. In some embodiments, performing the set of quantum superposition operations comprises: identifying a group of base pairs associated with the genomic scan data object; for each base pair, determining, based at least in part on applying a Hadamard quantum logic gate transformation of a pair of nucleobase features described by the base pair, a superposed representation; and determining the group of genome scan qubits based at least in part on each superposed representation.

At step/operation 503, the predictive data analysis computing entity 106 performs a set of constructive interference determination operations across the group of genome scan qubits to determine the one or more significant epistatic interactions. In some embodiments, a single beneficial epistatic occurrence is modeled by constructive quantum interference, and similarly, a single deleterious epistatic occurrence is modelled by destructive quantum interference. In some embodiments, the noted assumptions are key assumptions for modeling epistasis via interference in a quantum computing scenario. As a concrete example, the magnitude of a single qubit modelling a beneficial epistatic occurrence may be negated by the magnitude of a single qubit representing a deleterious epistatic occurrence.

In some embodiments, to perform the west of constructive interference determination operations, the predictive data analysis computing entity 106 chooses n, the order of epistatic interaction to be modelled. In some embodiments, the predictive data analysis computing entity 106 may be configured to restrict to, e.g., n={1, 2, 3}, to test the framework. This incorporates pairwise epistasis between any two loci, and further also includes epistatic interactions between any three loci. In some embodiments, to perform the west of constructive interference determination operations, the predictive data analysis computing entity 106 computes every n-mer of interactions between relevant loci, guided by existing (i.e., known) epistatic interactions. This is not typically possible on a conventional computer, but is ideal for implementation on a quantum computing platform. Additionally, two more methods of encoding and processing information exist in a qubit as a result to the fact that quantum computation uses amplitudes, which are a combination of magnitudes and phases. Qubits can therefore encode information in and compute with magnitudes and phases, which adds further expressive power to represent complex computational problems.

In some embodiments, a constructive interference determination is configured to determine, for each defined epistatic interaction between a defined group of two or more location-specific nucleobase features, whether the defined epistatic interaction corresponds to a constructive quantum interference or a destructive quantum interference. In some embodiments, as part of a constructive interference determination operation that is associated with a defined epistatic interaction, if the defined epistatic interaction is deemed a beneficial epistatic occurrence, it is modeled by a constructive quantum interference, while if the defined epistatic interaction is deemed a deleterious epistatic interaction, it is modeled by a destructive quantum interference. For example, in some embodiments, when performing a constructive interference determination operation with respect to an epistatic interaction that is characterized by presence of a guanine (G) nucleobase in a first defined locus and an adenine (A) nucleobase in a second defined locus, if the presence of the G nucleobase in the first defined locus along with the simultaneous presence of the A nucleobase in the second defined locus is deemed to increase the risk of a disease occurrence, then the epistatic interaction may be modeled using a constructive quantum interference; otherwise, the epistatic interaction may be modeled using a destructive quantum interference. In some embodiments, a constructive interference determination operation is configured to process the qubits corresponding to a corresponding epistatic interaction in the following manner to determine whether the epistatic interaction corresponds to a constructive quantum interference or a destructive quantum interference: perform quantum computing operations are performed to determine the output of an interference between the qubits corresponds to the corresponding epistatic interaction and then determine whether the output is a constructive quantum interference or a destructive quantum interference.

For example, in some embodiments, when performing a constructive interference determination operation with respect to an epistatic interaction that is characterized by presence of a thymine (T) nucleobase in a first defined locus and a guanine (G) nucleobase in a second defined locus, quantum computing operations are performed to determine the output of an interference between a qubit representation of the T nucleobase in the first defined locus and a qubit representation of the G nucleobase in the second defined locus, and then the noted output is used to determine whether the output is a constructive quantum interference or a destructive quantum interference. In some embodiments, quantum computing operations configured to perform interference between qubits use amplitudes (e.g., special combination of phases and magnitudes), which enable encoding additional information into qubits and further enable creating quantum digital and quantum phase logic that leverages interference for solving large complex problems.

In some embodiments, the set of quantum interference determination operations are performed by a maximal epistatic interaction order hyper-parameter that is configured to describe sizes of location-specific nucleobase features involved in epistatic interactions that may be deemed to be significant epistatic interactions for an epistatic interaction score generation sub-model of an epistatic polygenic risk score generation machine learning model.

For example, if the maximal epistatic interaction order hyper-parameter is three, then the significant epistatic interactions may include pairwise epistatic interactions between pairs of location-specific nucleobase features (e.g., a pair of location-specific nucleobase features that is characterized by presence of a guanine (G) nucleobase in a first defined locus and an adenine (A) nucleobase in a second defined locus), as well as triplet epistatic interactions between triplets of location-specific nucleobase features (e.g., a triplet of location-specific nucleobase features that is characterized by presence of a thymine (T) nucleobase in a first defined locus, a guanine (G) nucleobase in a second defined locus, and a cytosine (C) nucleobase in a third defined locus). In some embodiments, when determining the significant epistatic interactions for an epistatic interaction score generation sub-model of an epistatic polygenic risk score generation machine learning model, a training engine is configured to map defined groupings of n location-specific nucleobase features to a quantum space in order to perform constructive interference determination operations on each of the groupings, where n={1, . . . , h} and where h is the maximal epistatic interaction order hyper-parameter.

Determining significant epistatic interactions using constructive interference determination operations enables efficient identification of higher-order epistatic interactions, which as described above is a major technical challenge facing the field of computational genomics. See, e.g., T. McKay & J. Moore, “Why Epistasis Is Important for Tacking Complex Human Disease Genetics,” 6 Genome Medicine 42 (2014) (available online at https://genomemedicine.biomedcentral.com/articles/10.1186/gm561) (“The challenges for detecting epistasis in human populations are threefold . . . Another challenge in the analysis of epistasis is computational, and lies in the number of central processing unit cycles that are required to enumerate all possible combinatorial models. In general, it is not possible to test all possible interactions among more than three SNPs at a time in a genome-wide scan. A final challenge is interpretation. High-order interactions with non-additive effects can be difficult to comprehend statistically and perhaps even harder to tie back to biology. Designing combinatorial experiments to validate epistasis models might be more difficult than the analytical challenges.”).

By determining significant epistatic interactions using constructive interference determination operations, various embodiments of the present invention address the above-noted challenges associated with computational complexity of integrating epistatic interactions into polygenic risk score computation by utilizing quantum computing techniques. For example, in some embodiments, an epistatic polygenic risk score generation machine learning model is characterized by a set of significant epistatic interactions, where the set of significant epistatic interactions are determined by: (i) performing a set of quantum superposition operations on a genomic scan data object to generate a group of genome scan qubits, and (ii) performing a set of constructive interference determination operations across the group of genome scan qubits to determine the one or more significant epistatic interactions. By utilizing the noted techniques, various embodiments of the present invention enable reducing the amount of time and/or computational operations needed to detect significant epistatic interactions and integrate those significant epistatic integrations into polygenic risk score generations. In some embodiments, once identified and integrated into polygenic risk score generation models, epistatic interactions can be computed in an efficient manner, such as in linear time.

At step/operation 504, the predictive data analysis computing entity 106 generates an epistatic interaction score generation sub-model of the epistatic polygenic risk score generation machine learning model based at least in part on the set of significant epistatic interactions. In some embodiments, the epistatic interaction score generation sub-model defines an interaction weight for each significant epistatic interaction. In some of the noted embodiments, each interaction weight for a significant epistatic interaction is determined by generating a maximum likelihood estimation for the significant epistatic interaction using a maximum likelihood estimator for a normally distributed nonlinear implicit relationship defined by the epistatic polygenic risk score generation machine learning model. In some embodiments, epistatic interaction score generation sub-model of the epistatic polygenic risk score generation machine learning model may be configured to process one or more significant epistatic interaction features corresponding to one or more significant epistatic interactions to generate an epistatic interaction score for a patient data object.

The epistatic polygenic risk score generation machine learning model may be characterized by a set of significant epistatic interactions, where the set of significant epistatic interactions may be determined by: (i) performing a set of quantum superposition operations on a genomic scan data object to generate a group of genome scan qubits, and (ii) performing a set of constructive interference determination operations across the group of genome scan qubits to determine the one or more significant epistatic interactions.

At step/operation 505, the predictive data analysis computing entity 106 generates the epistatic polygenic risk score generation machine learning model based at least in part on the epistatic interaction score generation sub-model. In some embodiments, the epistatic polygenic risk score generation machine learning model comprises at least one of the following: (i) the epistatic interaction score generation sub-model that is configured to process one or more significant epistatic interaction features for the patient data object that correspond to one or more significant epistatic interactions defined by the epistatic interaction score generation sub-model in order to generate an epistatic interaction score, and (ii) a base polygenic risk score generation sub-model that is configured to process one or more significant genetic variant features for the patient data object that correspond to one or more significant genetic variants defined by the base polygenic risk score generation machine learning model in order to generate a base polygenic risk score.

In some embodiments, the epistatic polygenic risk score generation machine learning model comprises a linear equation having the form rₜₒₜₐₗ=θ₁r_{conv}Δθ₂rₑₚᵢₛₜₐₛᵢₛ, where rₜₒₜₐₗ is the epistatic polygenic risk score, r_{conv} is the base polygenic risk score, rₑₚᵢₛₜₐₛᵢₛ is the base epistatic interaction score, θ₁ and θ₂ are the respective weights for r_{conv} and rₑₚᵢₛₜₐₛᵢₛ (e.g., as determined using a maximum likelihood estimator for a normally distributed nonlinear implicit relationship defined by the epistatic polygenic risk score generation machine learning mode) and the operator A may have an algebraic form (e.g., may be one of addition, subtraction, multiplication , or division), which will be determined by iteratively cycling through the four possibilities and checking against hold-out data (e.g., positive cases of the disease in question obtained from the genome-wide association study result data).

Returning to FIG. 4, at step/operation 402, the predictive data analysis computing entity 106 generates the epistatic polygenic risk score using the epistatic polygenic risk score generation machine learning model. In some embodiments, generating the epistatic polygenic risk score using the epistatic polygenic risk score generation machine learning model comprises: (i) processing one or more significant epistatic interaction features corresponding to the one or more significant epistatic interactions using the epistatic interaction score generation sub-model to generate an epistatic interaction score, and (ii) generating the epistatic polygenic risk score based at least in part on the epistatic interaction score. In some embodiments, generating the epistatic polygenic risk score using the epistatic polygenic risk score generation machine learning model further comprises: (i) processing one or more significant genetic variant features corresponding to the one or more significant genetic variants using the base polygenic risk score generation sub-model to generate a base polygenic risk score, and (ii) generating the epistatic polygenic risk score based at least in part on the base polygenic risk score. In some embodiments, generating the epistatic polygenic risk score using the epistatic polygenic risk score generation machine learning model comprises: (i) processing one or more significant epistatic interaction features corresponding to the one or more significant epistatic interactions using the epistatic interaction score generation sub-model to generate an epistatic interaction score, (ii) processing one or more significant genetic variant features corresponding to the one or more significant genetic variants using the base polygenic risk score generation sub-model to generate a base polygenic risk score, and (iii) generating the epistatic polygenic risk score based at least in part on the base polygenic risk score and the epistatic interaction score.

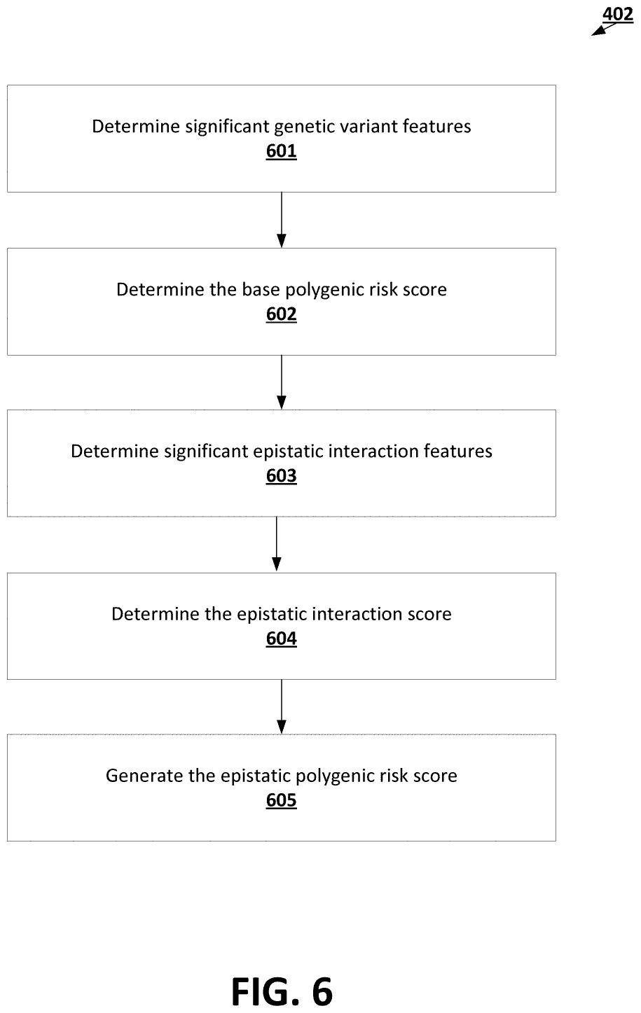

In some embodiments, step/operation 402 may be performed in accordance with the process that is depicted in FIG. 6. The process that is depicted in FIG. 6 begins at step/operation 601 when the predictive data analysis computing entity 106 processes the genomic scan data object for a patient data object to determine one or more significant genetic variant features. In some embodiments, each significant variant genetic feature may describe whether the genomic scan data object indicates presence of a significant genetic variant that is associated with the significant genetic variant feature. For example, a particular significant genetic variant feature may describe whether the genomic scan data object describes presence of a thymine (T) nucleobase in a particular defined locus, where presence of the T nucleobase in the particular defined locus may be deemed to be significant to occurrence probability of a condition/disease of interest.

At step/operation 602, the predictive data analysis computing entity 106 determines the base polygenic risk score based at least in part on the significant genetic variant features. In some embodiments, the predictive data analysis computing entity 106 processes the significant genetic variant features using the base polygenic risk score generation sub-model of the epistatic polygenic risk score generation machine learning model to generate the base polygenic risk score. In some embodiments, the base polygenic risk score generation sub-model of the epistatic polygenic risk score generation machine learning model defines, for each significant genetic variant, a weight, such as a weight that is determined using at least one of LD adjustment, Beta shrinkage, and P-value threshold across data describing correlations between genetic variants and disease occurrences as determined based at least in part on results of a genome-wide association study data.

In some embodiments, the base polygenic risk score describes an inferred risk that a patient data object describing a patient may be predicted to in the future suffer from a disease described by a disease data object, where the inferred risk is determined based at least in part on individual genetic variant features described by the genomic scan data object of the patient data object. In some embodiments, the base polygenic risk score describes how much individual genetic variant features described by the genomic scan data object of the patient data object suggest that the patient associated with the patient data object may be predicted to in the future suffer from the disease described by the disease data object. In some embodiments, the base polygenic risk score is a conventional Polygenic Risk Score value that is determined using a Polygenic Risk Score equation for the disease data object, where the Polygenic Risk Score equation may be determined by performing linkage disequilibrium (LD) adjustment, Beta shrinkage, and P-value threshold across data describing correlations between genetic variants and disease occurrences as determined based at least in part on results of a genome-wide association study data. In some embodiments, the base polygenic risk score describes whether the genomic scan data object associated with the patient data object describes each of a set of significant genetic variants as described by a base polygenic risk score generation sub-model of an epistatic polygenic risk score generation machine learning model.

At step/operation 603, the predictive data analysis computing entity 106 processes the genomic scan data object to determine one or more significant epi static interaction features. In some embodiments, each significant epistatic interaction feature describes whether the genomic scan data object indicates presence of n defined nucleobases in n defined loci, where the presence of the n defined nucleobases in the n defined loci may be deemed to be significant to occurrence probability of a condition/disease of interest. For example, a particular significant epistatic interaction feature may describe whether the genomic scan data object describes presence of a thymine (T) nucleobase in a first defined locus, a guanine (G) nucleobase in a second defined locus, and a cytosine (C) nucleobase in a third defined locus), where the presence of the T nucleobase in the first defined locus, the U nucleobase in the second defined locus, and the C nucleobase in the third defined locus may be deemed to be significant to occurrence probability of a condition/disease of interest.

At step/operation 604, the predictive data analysis computing entity 106 determines the epistatic interaction score based at least in part on the significant epistatic interaction features. In some embodiments, In some embodiments, the predictive data analysis computing entity 106 processes the significant epistatic interaction features using the epistatic polygenic risk score generation sub-model of the epistatic polygenic risk score generation machine learning model to generate the epistatic interaction score. In some embodiments, the epistatic interaction score generation sub-model defines an interaction weight for each significant epistatic interaction, where the interaction weight for a significant epistatic interaction may be determined by generating a maximum likelihood estimation for the significant epistatic interaction using a maximum likelihood estimator for a normally distributed nonlinear implicit relationship defined by the epistatic polygenic risk score generation machine learning model, for example using the maximum likelihood estimators described in G. R. Dolby and S. Lipton, Maximum Likelihood Estimation of the General Nonlinear Functional Relationship with Replicated Observations and Correlated Errors, Biometrika, Vol. 59, No. 1 (April, 1972), pp. 121-129 (available online at https://projecteuclid.org/download/pdf_1/euclid.aos/1176350696)).

In some embodiments, the epistatic interaction score describes an inferred risk that a patient data object describing a patient may be predicted to in the future suffer from a disease described by a disease data object, where the inferred risk is determined based at least in part on epistatic interactions across n-groupings of location-specific nucleobase features described in defined loci of a genomic scan data object of the patient data object. In some embodiments, the epistatic interaction score describes how much n-groupings of location-specific nucleobase features described in defined loci of a genomic scan data object of the patient data object suggest that the patient associated with the patient data object may be predicted to in the future suffer from the disease described by the disease data object. In some embodiments, the epistatic interaction score is determined based at least in part on whether the genomic scan data object associated with the patient data object describes each of a set of significant epistatic interactions, where the set of significant epistatic interactions may be defined by an epistatic interaction score generation sub-model of an epistatic polygenic risk score generation machine learning model.

At step/operation 605, the predictive data analysis computing entity 106 determines the epistatic polygenic risk score based at least in part on the base polygenic risk score and the epistatic interaction score. In some embodiments, the predictive data analysis computing entity 106 generates the epistatic polygenic risk score by combining the base polygenic risk score and the epistatic interaction score in accordance with the operations defined by the linear equation having the form rₜₒₜₐₗ=θ₁r_{conv}Δθ₂rₑₚᵢₛₜₐₛᵢₛ, where rₜₒₜₐₗ is the epistatic polygenic risk score, r_{conv} is the base polygenic risk score, rₑₚᵢₛₜₐₛᵢₛ is the base epistatic interaction score, θ₁ and θ₂ are the respective weights for r_{conv} and rₑₚᵢₛₜₐₛᵢₛ (e.g., as determined using a maximum likelihood estimator for a normally distributed nonlinear implicit relationship defined by the epistatic polygenic risk score generation machine learning model, for example using the maximum likelihood estimators described in G. R. Dolby and S. Lipton, Maximum Likelihood Estimation of the General Nonlinear Functional Relationship with Replicated Observations and Correlated Errors, Biometrika, Vol. 59, No. 1 (April, 1972), pp. 121-129 (available online at https://projecteuclid.org/download/pdf_1/euclid.aos/1176350696)), and the operator A may have an algebraic form (e.g., may be one of addition, subtraction, multiplication , or division), which will be determined by iteratively cycling through the four possibilities and checking against hold-out data (e.g., positive cases of the disease in question obtained from the genome-wide association study result data).

In some embodiments, the epistatic polygenic risk score describes an inferred risk that a patient data object describing a patient may be predicted to in the future suffer from a disease described by a disease data object. The epistatic polygenic risk score may be determined based at least in part on an epistatic interaction score that describes how much n-groupings of location-specific nucleobase features described in defined loci of a genomic scan data object of the patient data object suggest that the patient associated with the patient data object may be predicted to in the future suffer from the disease described by the disease data object.

In some embodiments, the epistatic polygenic risk score is determined based at least in part on both of: (i) a base polygenic risk score that describes how much individual genetic variant features described by the genomic scan data object of the patient data object suggest that the patient associated with the patient data object may be predicted to in the future suffer from the disease described by the disease data object, and (ii) an epistatic interaction score that describes how much n-groupings of location-specific nucleobase features described in defined loci of a genomic scan data object of the patient data object suggest that the patient associated with the patient data object may be predicted to in the future suffer from the disease described by the disease data object. For example, in some embodiments, the epistatic polygenic risk score is determined based at least in part on addition of the base polygenic risk score and the epistatic interaction score. As another example, in some embodiments, the epistatic polygenic risk score is determined based at least in part on a defined linear combination of the base polygenic risk score and the epistatic interaction score.

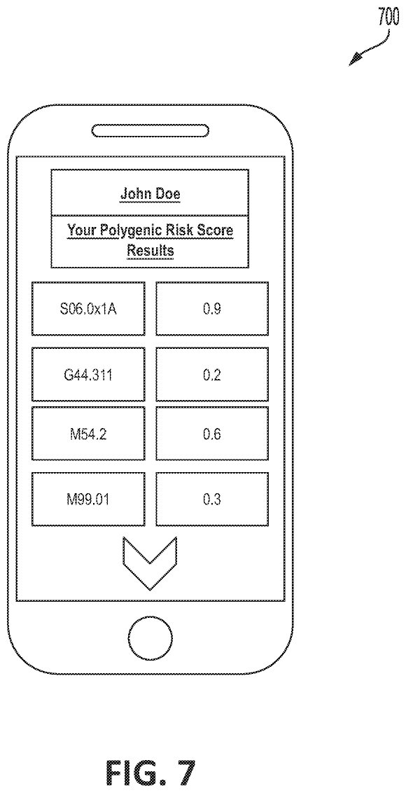

Returning to FIG. 4, at step/operation 403, the predictive data analysis computing entity 106 performs one or more prediction-based actions based at least in part on the epistatic polygenic risk score. Examples of prediction-based actions including displaying a user interface that displays health-related risk predictions (e.g., at least one of epistatic polygenic risk scores, epistatic interaction scores, and base polygenic risk scores) for a target individual with respect to a set of conditions. For example, as depicted in FIG. 7, the predictive output user interface 700 depicts the health-related risk prediction for a target individual with respect to four target conditions each identified by the International Statistical Classification of Diseases and Related Health Problems (ICD) code of the noted four target conditions.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for generating an epistatic polygenic risk score, the computer-implemented method comprising:
identifying, using one or more processors, an epistatic polygenic risk score generation machine learning model, wherein:
the epistatic polygenic risk score generation machine learning model comprises an epistatic polygenic risk score generation sub-model,
the epistatic polygenic risk score generation sub-model is characterized by one or more significant epistatic interactions, and
the one or more significant epistatic interactions are determined by: (i) performing a set of quantum superposition operations on a genomic scan data object to generate a group of genome scan qubits, and (ii) performing a set of constructive interference determination operations across the group of genome scan qubits to determine the one or more significant epistatic interactions;
generating, using the one or more processors and the epistatic polygenic risk score generation machine learning model, the epistatic polygenic risk score, wherein generating the epistatic polygenic risk score using the epistatic polygenic risk score generation machine learning model comprises: (i) processing one or more significant epistatic interaction features corresponding to the one or more significant epistatic interactions using the epistatic polygenic risk score generation sub-model to generate an epistatic interaction score, and (ii) generating the epistatic polygenic risk score based at least in part on the epistatic interaction score, and
performing, using the one or more processors, one or more prediction-based actions based at least in part on the epistatic polygenic risk score.

2. The computer-implemented method of claim 1, wherein the epistatic polygenic risk score generation sub-model defines an interaction weight for each significant epistatic interaction.

3. The computer-implemented method of claim 2, wherein each interaction weight for a significant epistatic interaction is determined by generating a maximum likelihood estimation for the significant epistatic interaction using a maximum likelihood estimator for a normally distributed nonlinear implicit relationship defined by the epistatic polygenic risk score generation machine learning model.

4. The computer-implemented method of claim 1, wherein the epistatic polygenic risk score generation machine learning model further comprises a base polygenic risk score generation sub-model that is characterized by one or more significant genetic variants.

5. The computer-implemented method of claim 4, wherein generating the epistatic polygenic risk score using the epistatic polygenic risk score generation machine learning model further comprises:
processing one or more significant genetic variant features corresponding to the one or more significant genetic variants using the base polygenic risk score generation sub-model to generate a base polygenic risk score, and
generating the epistatic polygenic risk score based at least in part on the base polygenic risk score and the epistatic interaction score.

6. The computer-implemented method of claim 1, wherein performing the set of quantum superposition operations comprises:
identifying a group of loci associated with the genomic scan data object;
for each locus, determining, based at least in part on applying a Hadamard quantum logic gate transformation of a nucleobase feature described by the locus, a superposed representation; and
determining the group of genome scan qubits based at least in part on each superposed representation.

7. The computer-implemented method of claim 1, wherein the set of constructive interference determination operations are performed based at least in part on a maximal epistatic interaction order hyper-parameter of the epistatic polygenic risk score generation machine learning model.

8. An apparatus for generating an epistatic polygenic risk score, the apparatus comprising at least one processor and at least one memory including program code, the at least one memory and the program code configured to, with the processor, cause the apparatus to at least:
identify an epistatic polygenic risk score generation machine learning model, wherein:
the epistatic polygenic risk score generation machine learning model comprises an epistatic polygenic risk score generation sub-model,
the epistatic polygenic risk score generation sub-model is characterized by one or more significant epistatic interactions, and
the one or more significant epistatic interactions are determined by: (i) performing a set of quantum superposition operations on a genomic scan data object to generate a group of genome scan qubits, and (ii) performing a set of constructive interference determination operations across the group of genome scan qubits to determine the one or more significant epistatic interactions;
generate, using the epistatic polygenic risk score generation machine learning model, the epistatic polygenic risk score, wherein generating the epistatic polygenic risk score using the epistatic polygenic risk score generation machine learning model comprises: (i) processing one or more significant epistatic interaction features corresponding to the one or more significant epistatic interactions using the epistatic polygenic risk score generation sub-model to generate an epistatic interaction score, and (ii) generating the epistatic polygenic risk score based at least in part on the epistatic interaction score, and
perform one or more prediction-based actions based at least in part on the epistatic polygenic risk score.

9. The apparatus of claim 8, wherein the epistatic polygenic risk score generation sub-model defines an interaction weight for each significant epistatic interaction.

10. The apparatus of claim 9, wherein each interaction weight for a significant epistatic interaction is determined by generating a maximum likelihood estimation for the significant epistatic interaction using a maximum likelihood estimator for a normally distributed nonlinear implicit relationship defined by the epistatic polygenic risk score generation machine learning model.

11. The apparatus of claim 8, wherein the epistatic polygenic risk score generation machine learning model further comprises a base polygenic risk score generation sub-model that is characterized by one or more significant genetic variants.

12. The apparatus of claim 11, wherein generating the epistatic polygenic risk score using the epistatic polygenic risk score generation machine learning model further comprises:
processing one or more significant genetic variant features corresponding to the one or more significant genetic variants using the base polygenic risk score generation sub-model to generate a base polygenic risk score, and
generating the epistatic polygenic risk score based at least in part on the base polygenic risk score and the epistatic interaction score.

13. The apparatus of claim 8, wherein performing the set of quantum superposition operations comprises:
identifying a group of loci associated with the genomic scan data object;
for each locus, determining, based at least in part on applying a Hadamard quantum logic gate transformation of a nucleobase feature described by the locus, a superposed representation; and
determining the group of genome scan qubits based at least in part on each superposed representation.

14. The apparatus of claim 8, wherein the set of constructive interference determination operations are performed based at least in part on a maximal epistatic interaction order hyper-parameter of the epistatic polygenic risk score generation machine learning model.

15. A computer program product for generating an epistatic polygenic risk score, the computer program product comprising at least one non-transitory computer-readable storage medium having computer-readable program code portions stored therein, the computer-readable program code portions configured to:
identify an epistatic polygenic risk score generation machine learning model, wherein:
the epistatic polygenic risk score generation machine learning model comprises an epistatic polygenic risk score generation sub-model,
the epistatic polygenic risk score generation sub-model is characterized by one or more significant epistatic interactions, and
the one or more significant epistatic interactions are determined by: (i) performing a set of quantum superposition operations on a genomic scan data object to generate a group of genome scan qubits, and (ii) performing a set of constructive interference determination operations across the group of genome scan qubits to determine the one or more significant epistatic interactions;
generate, using the epistatic polygenic risk score generation machine learning model, the epistatic polygenic risk score, wherein generating the epistatic polygenic risk score using the epistatic polygenic risk score generation machine learning model comprises: (i) processing one or more significant epistatic interaction features corresponding to the one or more significant epistatic interactions using the epistatic polygenic risk score generation sub-model to generate an epistatic interaction score, and (ii) generating the epistatic polygenic risk score based at least in part on the epistatic interaction score, and
perform one or more prediction-based actions based at least in part on the epistatic polygenic risk score.

16. The computer-implemented method of claim 15, wherein the epistatic polygenic risk score generation sub-model defines an interaction weight for each significant epistatic interaction.

17. The computer-implemented method of claim 16, wherein each interaction weight for a significant epistatic interaction is determined by generating a maximum likelihood estimation for the significant epistatic interaction using a maximum likelihood estimator for a normally distributed nonlinear implicit relationship defined by the epistatic polygenic risk score generation machine learning model.

18. The computer-implemented method of claim 15, wherein the epistatic polygenic risk score generation machine learning model further comprises a base polygenic risk score generation sub-model that is characterized by one or more significant genetic variants.

19. The computer-implemented method of claim 18, wherein generating the epistatic polygenic risk score using the epistatic polygenic risk score generation machine learning model further comprises:
processing one or more significant genetic variant features corresponding to the one or more significant genetic variants using the base polygenic risk score generation sub-model to generate a base polygenic risk score, and
generating the epistatic polygenic risk score based at least in part on the base polygenic risk score and the epistatic interaction score.

20. The computer-implemented method of claim 15, wherein performing the set of quantum superposition operations comprises:
identifying a group of loci associated with the genomic scan data object;
for each locus, determining, based at least in part on applying a Hadamard quantum logic gate transformation of a nucleobase feature described by the locus, a superposed representation; and
determining the group of genome scan qubits based at least in part on each superposed representation.
